(19) United States
(12) Patent Application Publication
(10) Pub. No.: US 2024/0296922 A1
(43) Pub. Date: Sep. 5, 2024

(54) USING A GAN FOR ELECTRONIC HEALTH RECORD EXTRAPOLATION

(71) Applicant: INTERNATIONAL BUSINESS MACHINES CORPORATION, ARMONK, NY (US)

(72) Inventors: VADIM RATNER, Haifa (IL); Yoel Shoshan, Haifa (IL)

(21) Appl. No.: 18/177,199

(22) Filed: Mar. 2, 2023

(51) Int. Cl. G16H 10/60; G06N 3/08

(52) U.S. Cl. CPC G16H 10/60; G06N 3/08

(57) ABSTRACT

A method of using a generative adversarial network (GAN) for EHR extrapolation is provided. The method includes obtaining EHRs for a plurality of patients. A generative component of the GAN is used to generate artificial patient trajectories for a disease that are marked as real by a discriminative component of the GAN based on the obtained EHRs. The artificial patient trajectories for the disease that are marked as real are used to iteratively train the GAN. The trained GAN is applied to a new patient EHR to predict at least one hypothetical patient trajectory or latent diagnosis for the disease.

### BACKGROUND

Exemplary embodiments of the present inventive concept relate to electronic health record extrapolation, and more particularly, to using a generative adversarial network (GAN) for electronic health record (EHR) extrapolation.

Several thousand diseases can affect human beings. Diagnostic models (supervised and unsupervised) have been developed to facilitate disease diagnosis. However, these diagnostic models have practical shortcomings. Supervised diagnostic models require costly and tedious expert input and are only trained to detect an overt constellation of disease symptoms rather than subtle preliminary predictors that may not be apparent from singular specialist records. However, once this trained constellation of overt disease symptoms manifests in a new patient, the disease is often already at an advanced stage, thus hindering a patient's prognosis. These supervised diagnostic models are also developed using a limited EHR. For example, disease specific EHRs used by the experts are typically restricted to the date of formal diagnosis onward and/or limited by disease rarity. On the other hand, although unsupervised diagnostic models may be trained using comprehensive EHRs and cluster analysis, the discovered cluster relationships are often trivial and not-disease specific.

### SUMMARY

Exemplary embodiments of the present inventive concept relate to a method, a computer program product, and a system for using a GAN for EHR extrapolation.

According to an exemplary embodiment of the present inventive concept, a method of using a generative adversarial network (GAN) for electronic health record (EHR) extrapolation is provided. The method includes obtaining EHRs for a plurality of patients. A generative component of the GAN is used to generate artificial patient trajectories for a disease that are marked as real by a discriminative component of the GAN based on the obtained EHRs. The artificial patient trajectories for the disease that are marked as real are used to iteratively train the GAN. The trained GAN is applied to a new patient EHR to predict at least one hypothetical patient trajectory or latent diagnosis for the disease.

According to an exemplary embodiment of the present inventive concept, a computer program product for using a generative adversarial network (GAN) for electronic health record (EHR) extrapolation is provided. The computer program product includes one or more computer-readable storage media and program instructions stored on the one or more non-transitory computer-readable storage media capable of performing a method. The method includes obtaining EHRs for a plurality of patients. A generative component of the GAN is used to generate artificial patient trajectories for a disease that are marked as real by a discriminative component of the GAN based on the obtained EHRs. The artificial patient trajectories for the disease that are marked as real are used to iteratively train the GAN. The trained GAN is applied to a new patient EHR to predict at least one hypothetical patient trajectory or latent diagnosis for the disease.

According to an exemplary embodiment of the present inventive concept, a computer system for using a generative adversarial network (GAN) for electronic health record (EHR) extrapolation is provided. The computer system includes one or more computer processors, one or more computer-readable storage media, and program instructions stored on the one or more of the computer-readable storage media for execution by at least one of the one or more processors capable of performing a method. The method includes obtaining EHRs for a plurality of patients. A generative component of the GAN is used to generate artificial patient trajectories for a disease that are marked as real by a discriminative component of the GAN based on the obtained EHRs. The artificial patient trajectories for the disease that are marked as real are used to iteratively train the GAN. The trained GAN is applied to a new patient EHR to predict at least one hypothetical patient trajectory or latent diagnosis for the disease.

### BRIEF DESCRIPTION OF THE DRAWINGS

The following detailed description, given by way of example and not intended to limit the exemplary embodiments solely thereto, will best be appreciated in conjunction with the accompanying drawings, in which:

FIG. 1 illustrates a schematic diagram of computing environment 100 including a GAN for EHR extrapolation program 150, in accordance with an exemplary embodiment of the present inventive concept.

FIG. 2 illustrates a block diagram of components included in the GAN for EHR extrapolation program 150, in accordance with an exemplary embodiment of the present invention.

FIG. 3 illustrates a flowchart of using a GAN for EHR extrapolation 300, in accordance with an exemplary embodiment of the present inventive concept.

It is to be understood that the included drawings are not necessarily drawn to scale/proportion. The included drawings are merely schematic examples to assist in understanding of the present inventive concept and are not intended to portray fixed parameters. In the drawings, like numbering may represent like elements.

### DETAILED DESCRIPTION

Exemplary embodiments of the present inventive concept are disclosed hereafter. However, it shall be understood that the scope of the present inventive concept is dictated by the claims. The disclosed exemplary embodiments are merely illustrative of the claimed system, method, and computer program product. The present inventive concept may be embodied in many different forms and should not be construed as limited to only the exemplary embodiments set forth herein. Rather, these included exemplary embodiments are provided for completeness of disclosure and to facilitate an understanding to those skilled in the art. In the detailed description, discussion of well-known features and techniques may be omitted to avoid unnecessarily obscuring the presented exemplary embodiments.

References in the specification to “one embodiment,” “an embodiment,” “an exemplary embodiment,” etc., indicate that the embodiment described may include a particular feature, structure, or characteristic, but not every embodiment may necessarily include that feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether explicitly described.

In the interest of not obscuring the presentation of the exemplary embodiments of the present inventive concept, in the following detailed description, some processing steps or operations that are known in the art may have been combined for presentation and for illustration purposes, and in some instances, may have not been described in detail. Additionally, some processing steps or operations that are known in the art may not be described at all. The following detailed description is focused on the distinctive features or elements of the present inventive concept according to various exemplary embodiments.

The present inventive concept provides for a method, system, and computer program product for using a GAN for EHR extrapolation. GANs are algorithmic architectures that use two neural networks, pitting one against the other (thus the “adversarial” aspect) to generate new, synthetic (i.e., artificial) instances of data that can pass for real data. GANs are used widely in synthetic image, video, and voice generation. The present inventive concept leverages the adversarial component of the GAN to generate artificial patient EHRs, such as by modifying real patient EHRs, in order to “trick” the discriminator component. The result is an increasingly discerning trained model of the GAN which can be used to supplement EHR repositories for diseases, diagnose actual or imminent disease using subtle (and perhaps previously unknown) medically relevant features, and/or detail hypothetical subsequent patient visits which would be concerning for a disease in light of the patient's existing EHR timeline.

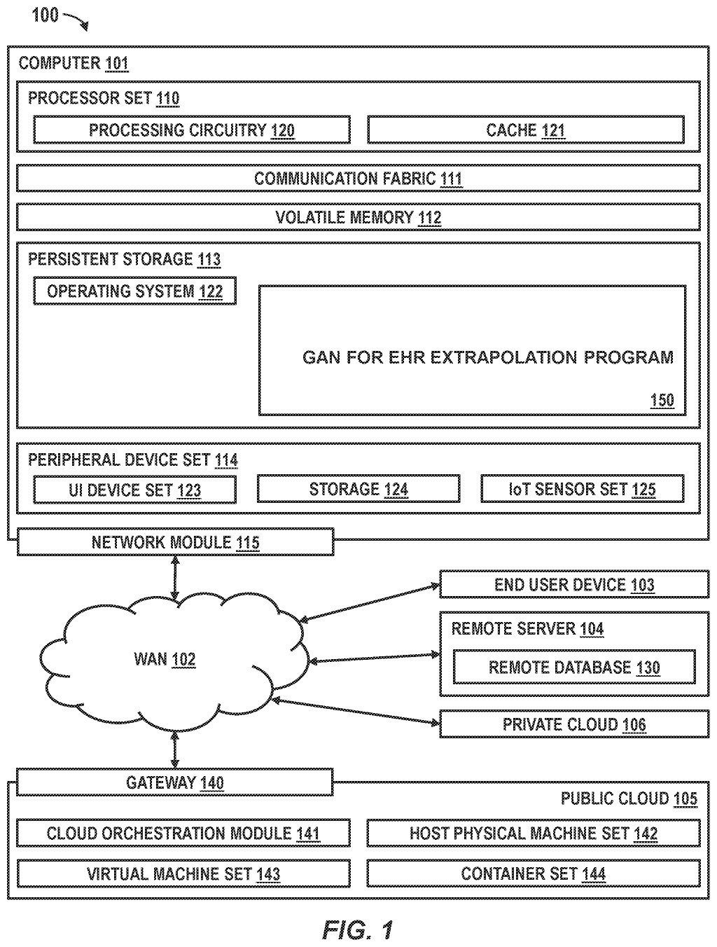

FIG. 1 illustrates a schematic diagram of computing environment 100 including a GAN for EHR extrapolation program 150, in accordance with an exemplary embodiment of the present inventive concept.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as a GAN for EHR extrapolation program 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 150 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

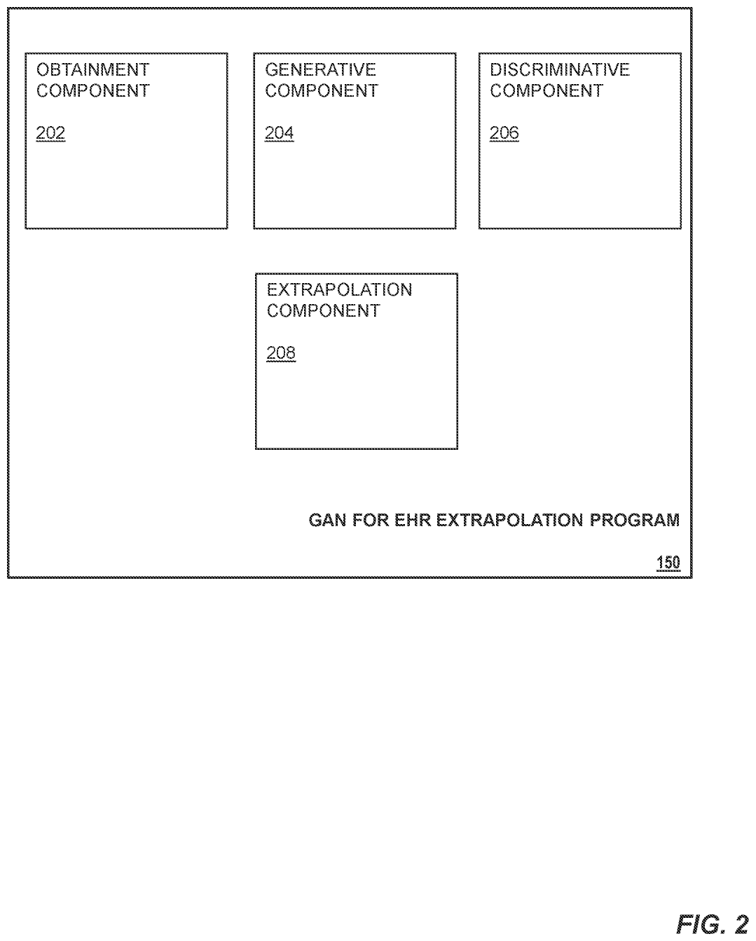

FIG. 2 illustrates a block diagram of components included in a GAN for EHR extrapolation program 150, in accordance with an exemplary embodiment of the present invention.

The EHR obtainment component 202 can obtain EHRs for a plurality of patients. The plurality of patients can include both healthy patients and ill patients (e.g., suffering from at least one disease). The obtained EHRs for each patient of the plurality of patients can be partial (e.g., limited by provider, specialty, medical group, hospital, patient complaint, time frame (e.g., disease onset-to-diagnosis), etc.) or can represent a comprehensive available EHR. The obtained EHRs for the ill patients can include patient trajectories for the at least one diagnosed disease. The EHR obtainment component 202 can obtain the EHRs for the plurality of patients via network access to at least one EHR repository (e.g., hospital record system, clinic database, unified health record service, etc.) and/or by user input (e.g., manual entry, multimedia upload (e.g., lab/image results, patient voicemails/portal messages), dictated clinician notes, etc.). The EHR obtainment component 202 can anonymize the obtained EHRs to protect patient privacy by removing personally identifying information (e.g., names, addresses, social security numbers, etc.). The EHR obtainment component 202 can extract relevant features from the obtained EHRs using AI processes (e.g., natural language processing (NLP), computer vision, text-to-speech transcription, etc.). Extracted features can include, but are not limited to, relevant medical terms, codes (e.g., international classification of diseases (ICD), current procedural terminology (CPT), etc.), administered treatments, prescriptions, prior personal/family histories, lab results, imaging results, examination results, known chemical exposures (e.g., occupational hazards), diagnoses, differential diagnoses, patient symptoms, and dates/timelines/frequencies for corresponding patient visits and communications.

For example, the EHR obtainment component 202 obtains EHRs from authorized access to a multidisciplinary medical group's patient EHR records. The medical group's patient EHR records are thus typically comprised of visits to various types of medical specialists each. Some of the multidisciplinary medical group's patients have been diagnosed with rheumatoid arthritis (RA). The EHR obtainment component 202 identifies patient diagnosed with RA and associated patient trajectories. The EHR obtainment component 202 extracts medically relevant features from the EHRs for the patients diagnosed with RA, such as bloodwork and chief complaints for all visits (not just with rheumatologists).

The GAN for EHR extrapolation program 150 can be preliminarily trained (e.g., in an unsupervised capacity) with the extracted features and associated dates/timelines/frequencies to obtain a general familiarity with the at least one disease and overt symptoms thereof. The generative component 204 can generate artificial patient trajectories related to the at least one disease identified in the obtained EHRs. The generative component 204 can generate the artificial patient trajectories by at least one of modifying, removing, and/or adding features and/or entire patient visits to a patient trajectory timeline for at least one patient (ill or healthy). Additionally, or alternatively, the generative component 204 can splice together artificial and/or real patient trajectories for the at least one disease (e.g., intermixing/substituting/adding visits associated with separate patients). The discriminative component 206 can engage in an iterative adversarial task with the generative component 204 in which the discriminative component 206 marks presented artificial patient trajectories as real, fake, and/or artificial. When the discriminative component 206 erroneously marks an artificial patient trajectory as real, it will self-refine by searching for increasingly nuanced clues indicative of authentic actual or imminent illness with the at least one disease. This adversarial task can be repeated until the discriminative component 206 accurately marks the artificial patient trajectories for the at least one disease within a predetermined threshold (e.g., accurate around 50% of the time).

For example, the preliminarily trained GAN for EHR extrapolation program 150 has a rudimentary understanding of RA's overt symptoms, such as a patient's presentation with threshold levels of rheumatoid factor and at least one joint deformity. The generative component 204 modifies the EHR for a healthy patient (i.e., without RA), initially adding the presence of peri-threshold levels of rheumatoid factor to numerous patient visits in the EHR timeline. Despite the addition of rheumatoid factor, the discriminative component 206 marks the artificial patient trajectory for RA as fake because the rheumatoid factor was not consistently detected, and the patient never complained of swelling, pain, tenderness, or stiffness in more than one joint, fatigue, symmetrical joint problems, otherwise unexplained fevers, or joint deformity. The generative component 204 then progressively modifies symptoms and detected rheumatoid factor frequency and levels in a multitude of permutations in an adversarial process with the discriminative component 206 until a near stalemate is reached.

The extrapolation component 208 can use the artificial patient trajectories that are marked as real to augment EHR repositories and analyse threshold features thereof. As a result, the trained GAN for EHR extrapolation program 150 will learn increasingly subtle and previously unknown clues (i.e., medically relevant features) that portend a latent disease diagnosis, but which may be attributed to seemingly unrelated patient visits. These clues and associated timelines thereof may be identified in the abstract for the at least disease. In addition, the extrapolation component 208 can identify confounding factors, comorbidities, and distinguish between diseases with similar presentations. The trained GAN for EHR extrapolation program 150 can identify a latent disease diagnosis for a newly obtained patient EHR and/or give a percentage probability of actual disease or imminent disease. The trained GAN for EHR extrapolation program 150 can also develop a hypothetical continuity of the new patient EHR which features constellations of medically relevant features that would coincide with actual disease or imminent disease. The GAN for EHR extrapolation program 150 can be applied to a new patient EHR to predict at least one latent patient trajectory or diagnosis for at least one disease. In an embodiment, new patient visits can be progressively compared to hypothetical patient trajectories that would culminate in at least one disease. Changes in risk probability for at least one disease can be automatically detected, calculated, and presented to a user of the GAN for EHR extrapolation program 150.

For example, not all patients with RA present with rheumatoid factor, which can complicate and delay RA treatment and diagnosis for a substantial minority of patients. In combination with characteristic symptoms, the trained GAN eventually recognizes that high inflammatory erythrocyte sedimentation rate (ESR) from non-rheumatological bloodwork, for example, has a high correspondence with RA even in the absence of rheumatoid factor when there is no other competing explanation. Thus, when a new patient comes in with a substantially similar constellation of features, the trained GAN may detect probable RA even when the diagnosis is elusive or would be hindered by myopic patient specialist visits.

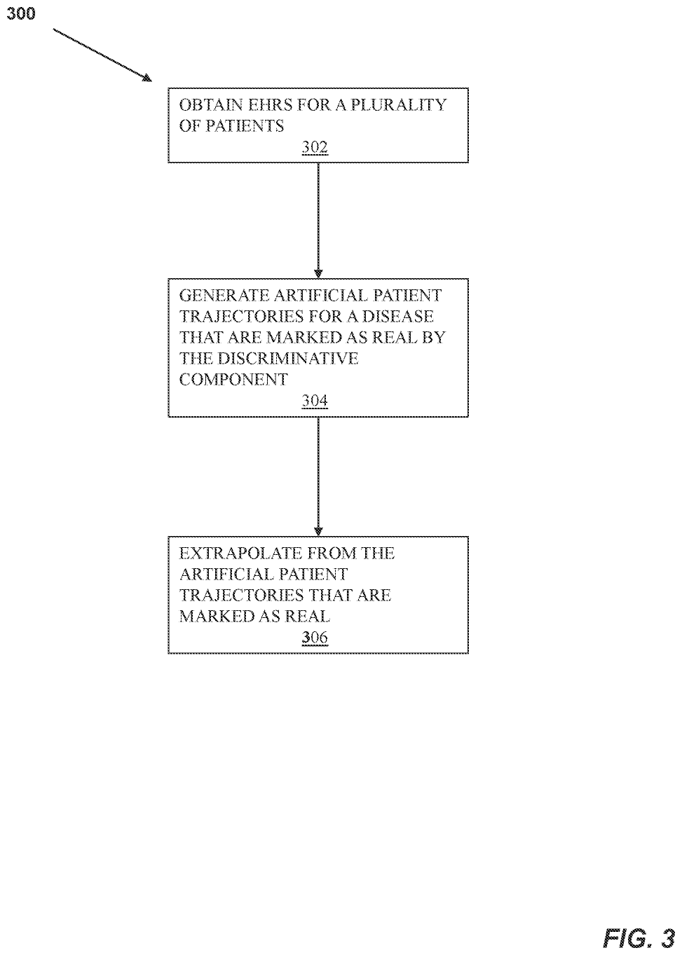

FIG. 3 illustrates a flowchart of using a GAN for EHR extrapolation 300, in accordance with an exemplary embodiment of the present inventive concept.

The GAN for EHR extrapolation program 150 can obtain EHRs for a plurality of patients using the obtainment component 201 (step 302).

The GAN for EHR extrapolation program 150 can use the generative component 204 to generate artificial patient trajectories for a disease that are marked as real by the discriminative component 206 (step 304).

The GAN for EHR extrapolation program 150 can extrapolate from the artificial patient trajectories that are marked as real using the extrapolation component 208 (step 306).

Based on the foregoing, a computer system, method, and computer program product have been disclosed. However, numerous modifications, additions, and substitutions can be made without deviating from the scope of the exemplary embodiments of the present inventive concept. Therefore, the exemplary embodiments of the present inventive concept have been disclosed by way of example and not by limitation.

1. A method of using a generative adversarial network (GAN) for electronic health record (EHR) extrapolation, the method comprising:
obtaining EHRs for a plurality of patients;
using a generative component of the GAN to generate artificial patient trajectories for a disease that are marked as real by a discriminative component of the GAN, based on the obtained EHRs;
using the artificial patient trajectories for the disease that are marked as real to train the GAN; and
applying the trained GAN to a new patient EHR to predict at least one hypothetical patient trajectory or latent diagnosis for the disease.

2. The method of claim 1, further comprising:
augmenting an EHR repository with the artificial patient trajectories for the disease that are marked as real.

3. The method of claim 1, further comprising:
identifying threshold diagnostic features of the disease using the artificial patient trajectories for the disease that are marked as real.

4. The method of claim 1, wherein the step for using the artificial patient trajectories for the disease that are marked as real to train the GAN is performed in an iterative adversarial manner.

5. The method of claim 1, wherein the generative component of the GAN generates the artificial patient trajectories by modifying at least one patient trajectory for the disease included in the obtained EHR.

6. The method of claim 5, wherein the modifying of the at least one patient trajectory for the disease includes manipulating at least one of a diagnosis, treatment, prescription, or symptom.

7. The method of claim 6, wherein the manipulating of the at least one of diagnosis, treatment, prescription, or symptom involves altering at least one actual patient visit or generating an artificial patient visit.

8. A computer program product for using a generative adversarial network (GAN) for electronic health record (EHR) extrapolation, the computer program product comprising:
one or more computer-readable storage media and program instructions stored on the one or more non-transitory computer-readable storage media capable of performing a method, the method comprising:
obtaining EHRs for a plurality of patients;
using a generative component of the GAN to generate artificial patient trajectories for a disease that are marked as real by a discriminative component of the GAN, based on the obtained EHRs;
using the artificial patient trajectories for the disease that are marked as real to train the GAN; and
applying the trained GAN to a new patient EHR to predict at least one hypothetical patient trajectory or latent diagnosis for the disease.

9. The computer program product of claim 8, further comprising:
augmenting an EHR repository with the artificial patient trajectories for the disease that are marked as real.

10. The computer program product of claim 8, further comprising:
identifying threshold diagnostic features of the disease using the artificial patient trajectories for the disease that are marked as real.

11. The computer program product of claim 8, wherein the step for using the artificial patient trajectories for the disease that are marked as real to train the GAN is performed in an iterative adversarial manner.

12. The computer program product of claim 8, wherein the generative component of the GAN generates the artificial patient trajectories by modifying at least one patient trajectory for the disease included in the obtained EHRs.

13. The computer program product of claim 12, wherein the modifying of the at least one patient trajectory for the disease includes manipulating at least one of diagnosis, treatment, prescription, or symptom.

14. The computer program product of claim 13, wherein the manipulating of the at least one of diagnosis, treatment, prescription, or symptom involves altering at least one actual patient visit or generating an artificial patient visit.

15. A computer system for using a generative adversarial network (GAN) for electronic health record (EHR) extrapolation, the computer system comprising:
one or more computer processors, one or more computer-readable storage media, and program instructions stored on the one or more of the computer-readable storage media for execution by at least one of the one or more processors capable of performing a method, the method comprising:
obtaining EHRs for a plurality of patients;
using a generative component of the GAN to generate artificial patient trajectories for a disease that are marked as real by a discriminative component of the GAN, based on the obtained EHRs;
using the artificial patient trajectories for the disease that are marked as real to train the GAN; and
applying the trained GAN to a new patient EHR to predict at least one hypothetical patient trajectory or latent diagnosis for the disease.

16. The computer system of claim 15, further comprising:
augmenting an EHR repository with the artificial patient trajectories for the disease that are marked as real.

17. The computer system of claim 15, further comprising:
identifying threshold diagnostic features of the disease using the artificial patient trajectories for the disease that are marked as real.

18. The computer system of claim 15, wherein the step for using the artificial patient trajectories for the disease that are marked as real to train the GAN is performed in an iterative adversarial manner.

19. The computer system of claim 15, wherein the generative component of the GAN generates the artificial patient trajectories by modifying at least one patient trajectory for the disease.

20. The computer system of claim 19, wherein the modifying of the at least one patient trajectory for the disease includes manipulating at least one of diagnosis, treatment, prescription, or symptom.
